(19) United States
(12) Patent Application Publication
(10) Pub. No.: US 2026/0232954 A1
(43) Pub. Date: Aug. 13, 2026

(54) URINE MANAGEMENT SYSTEMS

(71) Applicant: Sage Products, LLC, Cary, IL (US)

(72) Inventors: Sumit Dalal, New Delhi (IN); Brajesh Kumar, Uttar Pradesh (IN); Nitin Ahlawat, Delhi (IN); Pratyush Tiwari, Haryana (IN)

(73) Assignee: Sage Products, LLC, Cary, IL (US)

(21) Appl. No.: 19/538,795

(22) Filed: Feb. 12, 2026

(30) Foreign Application Priority Data
Feb. 13, 2025 (IN) 202541012156

(51) Int. Cl. A61M 25/00; A61F 5/451; A61M 1/00; G16H 40/63

(52) U.S. Cl. CPC A61M 25/0017; A61F 5/451; A61M 1/73; A61M 1/74; A61M 1/782; G16H 40/63; A61M 2202/0496; A61M 2205/18; A61M 2205/3351; A61M 2205/50

(57) ABSTRACT

A urine suction system includes a cannister with a first port, a first tube, and a pump system. The first tube is coupled to the first port and receives fluid from the first port. The pump system includes a pump inlet, a first sensor, a pump, a pump outlet, and a processing circuit. The pump inlet coupled to the first tube and receives the fluid from the first tube. The first sensor provides a signal associated with a pressure of the fluid within the first tube. The pump receives the fluid from the pump inlet. The pump outlet receives the fluid from the pump. The processing circuit includes a memory and one or more processors that receive a signal from the first sensor, determine the pressure based on the signal, and compare the pressure to a pump threshold, cause the pump to provide the fluid at a first rate.

### CROSS-REFERENCE TO RELATED PATENT APPLICATION

The application claims priority to and the benefit of Indian Provisional Patent Application No. 202541012156, filed Feb. 13, 2025, the disclosure of which is incorporated herein by reference in its entireties for all purposes.

### TECHNICAL FIELD

The present disclosure relates generally to urine management systems. More specifically, this application relates to systems for facilitating urine movement from a patient.

### BACKGROUND

Patients in medical facilities often suffer from various ailments or physical limitations. Such ailments or physical limitations commonly restrict urinary output of patients during treatment and observation. As a result, medical facilities may utilize catheters to assist with urinary output. Nurses and other caregivers at the medical facilities may often encounter difficulties in detecting presence of urine in a catheter, fill status of an external cannister, and obstructions in the tubing of the catheter.

### SUMMARY

Various embodiments relate to a urine suction system. The urine suction system includes a cannister, a first tube, and a pump system. The cannister includes a first port. The first tube is coupled to the first port and is configured to receive fluid from the first port. The pump system includes a pump inlet, a first sensor, a pump, a pump outlet, and a processing circuit. The pump inlet is coupled to the first tube and configured to receive the fluid from the first tube. The first sensor is configured to provide a signal associated with a pressure of the fluid within the first tube. The pump is configured to receive the fluid from the pump inlet. The pump outlet is configured to receive the fluid from the pump. The processing circuit includes a memory and one or more processors. The one or more processors are configured to receive a signal from the first sensor, determine the pressure based on the signal, and compare the pressure to a pump threshold, cause the pump to provide the fluid at a first rate when the pressure is less than the pump threshold, and cause the pump to provide the fluid at a second rate greater than the first rate when the pressure is greater than or equal to the pump threshold.

Various embodiments relate to a method of operating a urine suction system. The method includes receiving, by one or more processors, a signal from a first sensor. The signal associated with a pressure of a fluid within a pump tube coupled to a cannister and a pump. The method includes determining, by the one or more processors, a pressure value based on the signal. The method includes comparing, by the one or more processors, the pressure value to a pump threshold. The method includes instructing, by the one or more processors, the pump to provide the fluid at a first rate via the pump tube when the pressure value is less than the pump threshold. The method includes instructing, by the one or more processors, the pump to provide the fluid at a second rate greater than the first rate via the pump tube when the pressure value is greater than or equal to the pump threshold.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a block diagram of an example urine management system;

FIG. 2 is a system diagram of a portion of the urine management system of FIG. 1;

FIG. 3 is a view of another portion of the urine management system of FIG. 1;

FIG. 4 is a view of another portion of the urine management system of FIG. 1;

FIG. 5 is a top view of another portion of the urine management of FIG. 1;

FIG. 6 is a side view of a portion of a pump system;

FIG. 7 is a bottom view of another portion of the pump system;

FIG. 8 is a bottom view of another portion of the pump system;

FIG. 9 is a block diagram of a portion of a control system;

FIG. 10 is a top view of another portion of the pump system in a first state;

FIG. 11 is a top view of the portion of the pump system of FIG. 10 in a second state;

FIG. 12 is a top view of portion of the pump system of FIG. 10 in a third state;

FIG. 13 is a state diagram of operations of the urine management system after being powered on;

FIG. 14 is a block diagram of a portion of a process for operating a pump system of the urine management system;

FIG. 15 is a block diagram of another portion of the process of FIG. 14; and

FIG. 16 is a block diagram of another portion of the process of FIG. 14.

### DETAILED DESCRIPTION

Before turning to the figures, which illustrate certain example embodiments in detail, it should be understood that the present disclosure is not limited to the details or methodology set forth in the description or illustrated in the figures. It should also be understood that the terminology used herein is for the purpose of description only and should not be regarded as limiting. The various concepts introduced above and discussed in greater detail below may be implemented in any of a number of ways, as the described concepts are not limited to any particular manner of implementation. Examples of specific implementations and applications are provided primarily for illustrative purposes.

### I. Overview

Implementations described herein relate to a urine management system. The urine management system may be utilized in a hospital, clinic, assisted living, or other similar patient care setting. The urine management system includes a catheter and urine suction system. The urine suction system is configured to detect the presence of urine within a tube connected to the catheter during operation. For example, the urine suction system may ensure a continuous suction to pull all urine to a urine collection cannister during use with a patient. The urine suction system may be particularly beneficial for patients with decreased mobility, patient under ventilation, or patients with various medical conditions.

Additionally, the urine suction system may detect and monitor the fill status of the urine collection cannister. The urine suction system may mitigate a frequency in which a nurse manually (e.g., visually) monitors a fill status of the urine collection cannister. For example, the urine suction system may notify a nurse if the fill status of the urine collection cannister reaches operational capacity, which may prevent urine leakage and protect the patient. The urine suction system may be particularly beneficial for patients with severe medical conditions or patients in an unresponsive or sedated state.

Additionally, the urine suction system may detect obstructions in tubing of the urine management system. The urine suction system may mitigate injury by decreasing a speed of a motor of the urine suction system and alerting a nurse. For example, the urine suction system may notify the nurse if an obstruction is present, therefore protecting the patient from injury.

### II. Overview of Example Urine Management System

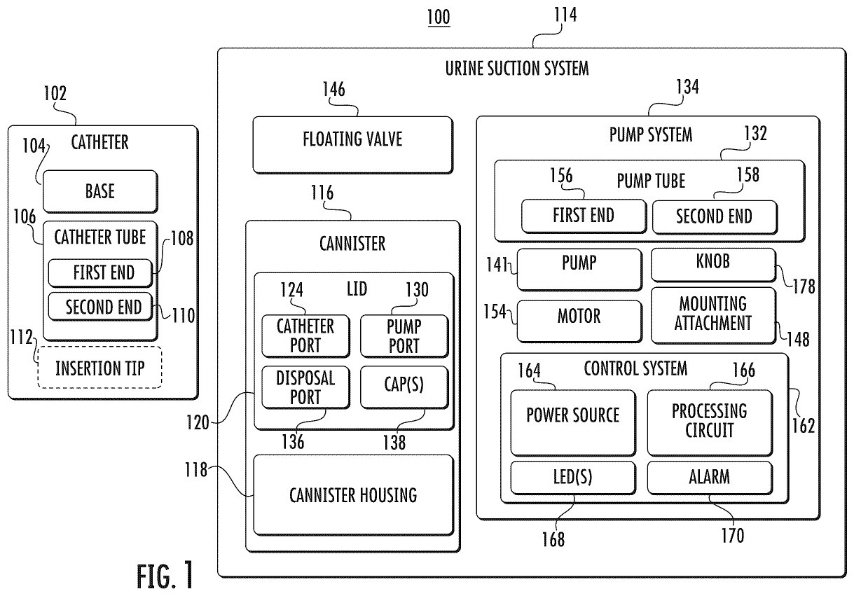

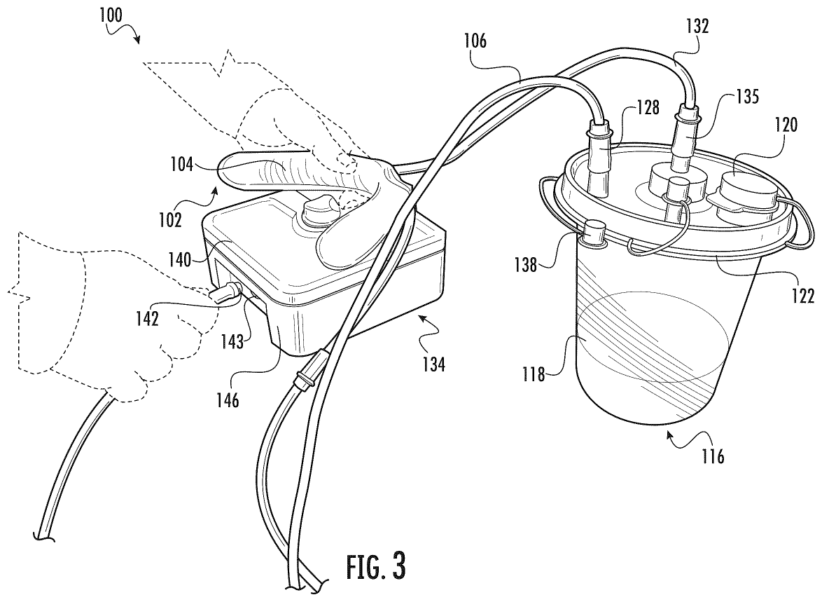

FIGS. 1-3 illustrate a urine management system 100 (e.g., system, treatment system, incontinence management system). As is explained in more detail below, the urine management system 100 may be utilized by a nurse (e.g., caretaker, assistant, doctor) to provide care for a patient (e.g., human, elderly person, person with a disability, ventilated person, person without a disability), such as a patient with limited mobility or illnesses involving urinary incontinence.

The urine management system 100 includes a catheter 102 (e.g., urinary catheter). In various embodiments, the catheter 102 is an external catheter (e.g., non-invasive catheter). In various embodiments, the catheter 102 may be a male external catheter or a female external catheter. For example, while the FIGURES depict the use of a female external catheter, the urine management system 100 may instead include an external catheter designed for male anatomy (e.g., male external catheter, retracted penis external catheter). The catheter 102 may be an indwelling catheter or a suprapubic catheter (e.g., invasive catheters). The catheter 102 is configured to facilitate drainage of urine from a patient due to various medical conditions (e.g., nerve damage, enlarged prostate, surgery). The catheter 102 may be configured to facilitate management of urinary incontinence. The catheter 102 may be configured to facilitate monitoring of fluid balance in patients with various medical conditions.

The catheter 102 includes a base 104. The base 104 is configured to be placed on the skin of a patient to secure the catheter 102 in place on the patient. For example, the base 104 may be placed over a portion, such as the glans (e.g., tip) of a penis of a male patient. As another example, the base 104 is positioned in the perineal area (e.g., between the vaginal opening and anus) of a female patient. In some embodiments, the base 104 is constructed of silicone, latex, polyurethane, or similar flexible materials. In other embodiments, the base 104 includes a silicone-based or hydrocolloid adhesive coating to secure the catheter 102 in place without causing skin irritation.

The catheter 102 includes a catheter tube 106. The catheter tube 106 includes a first end 108 and a second end 110. The first end 108 is configured to facilitate entrance of a fluid (e.g., urine, bodily fluids) from the base 104 into the catheter tube 106. The second end 110 is configured to expel the fluid into a disposal site (e.g., a urine collection bag, urine collection cannister, waste bin). The catheter tube 106 is coupled to the base 104 and the disposal site to facilitate flow of the fluid from the patient.

In some embodiments, such as where the catheter 102 is an indwelling catheter or a suprapubic catheter, the catheter 102 further includes an insertion tip 112. The insertion tip 112 includes a rounded end (not shown) that is configured to be inserted into the bladder of a patient. The insertion tip 112 is positioned at the first end 108. The insertion tip 112 is configured to guide the catheter 102 into the bladder of a patient to ensure proper placement. The insertion tip 112 is configured to receive fluid from the patient and facilitate entrance of the fluid into the catheter tube 106 at the first end 108. In some embodiments, the insertion tip 112 includes one or more drainage eyelets. The drainage eyelets may be placed near the tip to provide increased flow of fluid through the catheter tube 106.

As shown in FIG. 1, the urine management system 100 includes a urine suction system 114 (e.g., suction system). The urine suction system 114 is configured to provide suction of fluid from the patient and into a disposal site. The urine suction system 114 may be configured to provide suction of the fluid continuously (e.g., such that fluid is continuously suctioned from the patient). The urine suction system 114 may be configured to operate at low speeds when urine is absent, thereby reducing energy consumption and operational costs as well as reducing noise produced by components of the urine suction system 114. The urine suction system 114 is configured to adapt monitoring and detection for various occurrences (e.g., relaxation, urination, obstruction) during operation to prevent patient injury, as described in more detail below.

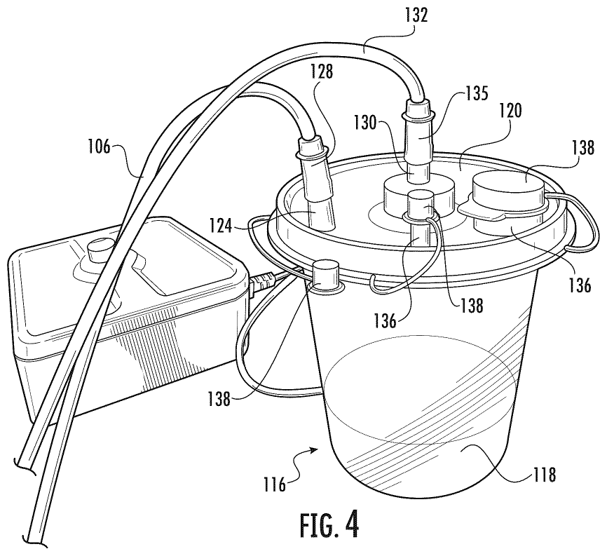

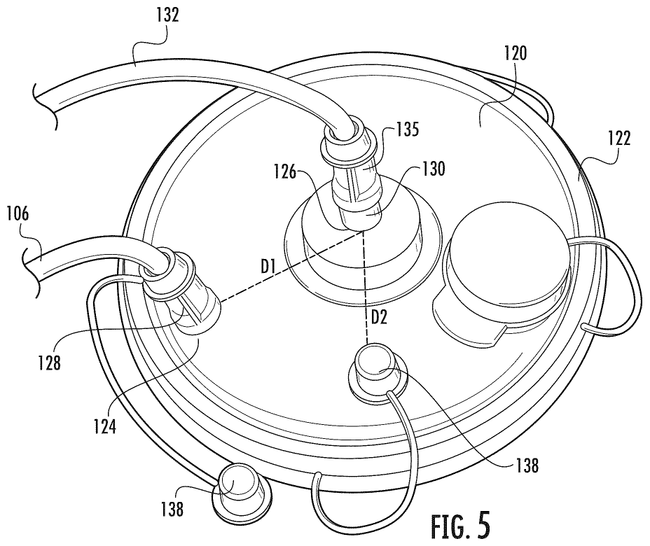

As shown in FIG. 1-5, the urine suction system 114 includes a cannister 116 (e.g., urine cannister, urine collection cannister). The cannister 116 is configured to capture and store fluid from the patient when connected to the catheter 102. The cannister 116 is connected to the catheter 102 via the catheter tube 106. The cannister 116 includes a cannister housing 118 and a lid 120.

The cannister housing 118 is configured to store fluid suctioned from the patient by the catheter 102. In various embodiments, the cannister housing 118 has a cylindrical shape. In some applications, the cannister housing 118 may have rectangular shape. In other applications, the cannister housing 118 may have a polygonal shape, configured to fit around various components of a hospital bed or hospital chair. In some embodiments, the cannister housing 118 is constructed of polypropylene, polyethylene, polyvinyl chloride, polyurethane, polystyrene, polycarbonate, silicone, and/or similar materials.

The lid 120 is configured to form a seal (e.g., fluid-tight seal) between the cannister housing 118 and lid 120. The lid 120 is formed in a similar shape as the cannister housing 118. For example, the lid 120 may have a cylindrical shape to fit on the cannister housing 118 with a cylindrical shape. In some applications, the lid 120 may include a threaded surface on an interior portion of the rim 122, where the threaded surface is configured to be threadably coupled to a threaded surface on the cannister housing 118. For example, the rim 122 may be screwed (e.g., twisted, fastened, locked) onto the cannister housing 118. In other applications, the lid 120 includes a silicone O-ring or a rubber gasket that is configured to create a seal between the cannister housing 118 and the lid 120. In other applications, the lid 120 is fused (e.g., welded) to the cannister housing 118 to prevent leakage. In some embodiments, the lid 120 is constructed of polypropylene, polyethylene, polyvinyl chloride, polyurethane, polystyrene, polycarbonate, silicone, or similar materials.

The lid 120 includes a catheter port 124 (e.g., second port). The catheter port 124 is cylindrical in shape. The catheter port 124 is positioned from a first distance D1 from a center 126 of the lid 120. The catheter port 124 is configured to receive a fluid from the patient via the catheter tube 106. The catheter tube 106 also includes a first adaptor 128, configured to secure the catheter tube 106 to the catheter port 124 to prevent leakage of fluid.

The lid 120 includes a pump port 130 (e.g., first port). In various embodiments, the pump port 130 is cylindrical in shape. The pump port 130 may be positioned at the center 126. The pump port 130 is configured to interact with a pump system 134 which creates suction pressure inside the cannister 116. The pump system 134 includes a pump tube 132, configured to connect the pump system 134 and the cannister 116, as will be described in more details below. The pump tube 132 includes a second adaptor 135, configured to secure the pump tube 132 to the pump port 130 to create a vacuum between the pump system 134 and the cannister 116.

The lid 120 includes a disposal port 136. In various embodiments, the disposal port 136 is cylindrical in shape. The disposal port 136 is positioned at a second distance D2 from the center 126. In some embodiments, the second distance D2 is equal to the first distance D1. In other embodiments, the second distance D2 is greater than the first distance D1. In other embodiments, the second distance D2 is less than the first distance D1. The disposal port 136 is configured to expel fluid from the cannister 116 into a separate disposal site (e.g., waste bin, storage container, toilet) when the cannister 116 is filled to operational capacity.

The lid 120 further includes one or more caps 138 positioned at the rim 122. Each of the one or more caps 138 is attached to the rim 122 via a thin strip of material. The one or more caps 138 are constructed with similar materials as the cannister housing 118 and the lid 120. For example, the one or more caps 138 may be constructed of polypropylene, polyethylene, polyvinyl chloride, polyurethane, polystyrene, polycarbonate, silicone, and/or similar materials. The one or more caps 138 are configured to prevent fluid leakage from cannister 116 via the catheter port 124, the pump port 130, and the disposal port 136. In some applications, the one or more caps 138 may include a threaded surface on an interior portion of the one or more caps 138, where the threaded surface is configured to be threadedly coupled to a threaded surface on the cannister housing 118. For example, the rim 122 may be screwed (e.g., twisted, fastened, locked) onto each respective port (e.g., catheter port 124, pump port 130, disposal port 136. In other applications, the one or more caps 138 includes a silicone O-ring or a rubber gasket that is configured to create a seal between each respective port and the one or more caps 138.

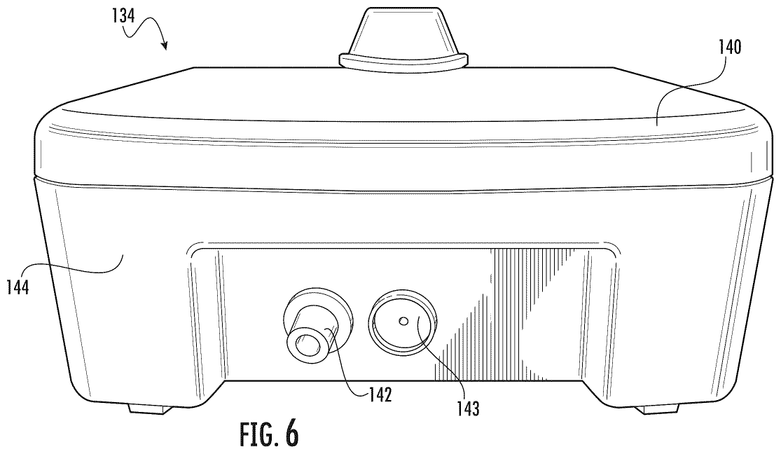

As shown in FIGS. 1 and 6-8, the urine suction system 114 includes a pump system 134. The pump system 134 is configured to adjust suction pressure during various periods of time. The pump system 134 is configured to function in a plurality of states (e.g., operating modes) including a normal state (e.g., pressure remains below the maximum safe threshold) and an obstruction state (e.g., pressure has risen above maximum safe threshold). The normal state further includes an active state (e.g., adjusting suction pressure when urine is detected), a sleep state (e.g., running the pump system 134 at low power when no urine is detected), and an obstruction state (e.g., pressure as risen above maximum threshold). The pump system 134 is configured to transition between these states to facilitate desirable suctioning of the fluid from the patient. For example, when the pump system 134 determines that a blockage has occurred, the pump system 134 may transition from the normal state to the obstruction state.

The pump system 134 includes a pump housing 140. The pump housing 140 is configured to enclose (e.g., protect, house) internal components of the pump system 134. The pump housing 140 is substantially rectangular in shape. In some applications, the pump housing 140 may be substantially polygonal in shape to accommodate the size and shape of internal components within the pump housing 140. In some embodiments, the pump housing 140 is constructed of medical-grade plastics such as polycarbonate, polypropylene, acrylic, or similar materials. In other embodiments, the pump housing is constructed of medical-grade metals such as stainless steel, aluminum, titanium, or similar materials.

The pump system 134 includes a pump 141. In various embodiments, the pump 141 is a diaphragm pump or vacuum pump. The pump 141 is disposed within the pump housing 140. The pump 141 is configured to create a pressure difference between the pump system 134 and the cannister 116, facilitating the flow of a fluid (e.g., air, gas) from the pump 141 to the cannister 116 via the pump tube 132.

The pump system 134 includes a pump inlet 142 and a pump outlet 143. The pump 141 is coupled to the pump inlet 142 and the pump outlet 143, where the pump 141 is configured to receive the fluid from the pump inlet 142. The pump inlet 142 and the pump outlet 143 are positioned on a first face 144 of the pump housing 140. The pump inlet 142 is coupled to the pump tube 132 and configured to receive the fluid from the pump tube 132. The pump outlet 143 is configured to receive the fluid from the pump 141 and expel the fluid from the pump system 134 at ambient or elevated pressure.

As shown in FIG. 1, the pump system 134 includes a floating valve 146 (e.g., self-regulating valve). The floating valve 146 is positioned between the pump 141 and pump inlet 142. The floating valve 146 is configured to (i) control flow of the fluid, (ii) control backflow of the fluid, and (iii) regulate pressure of the fluid within the urine suction system 114.

The floating valve 146 is configured to operate between a first position and a second position. The first position includes pump system 134 operating in the normal state, in which the floating valve 146 facilitates flow of the fluid from the pump inlet 142 to the pump 141. The second position includes the pump system 134 operating in the obstruction state, in which the first face 144 prohibits flow of the fluid from the pump inlet 142 to the pump 141 in instances where an obstruction is present or if the cannister 116 is filled at or above operational capacity. The floating valve 146 is configured to remain in the first position when a pressure of the pump tube 132 is greater than or equal to the valve threshold (e.g., the maximum safe threshold). The floating valve 146 is configured to transition into the second position when the pressure is less than the valve threshold. The valve threshold is determined by the volume of the cannister 116 connected to the pump system 134.

As shown in FIGS. 1, 7, and 8, the pump system 134 includes a mounting attachment 148 (e.g., clip, retainer). The mounting attachment 148 is positioned on a bottom face 150 of the pump housing 140. The mounting attachment 148 is positioned in a cavity 152 of the bottom face 150. In one application, the mounting attachment 148 is configured to mount the urine suction system 114 to an intravenous pole (e.g., IV pole, intravenous stand, infusion stand). In another application, the mounting attachment 148 is configured to mount the urine suction system 114 to another device within an intensive care unit room (e.g., the railing of a hospital bed, a chair). In some embodiments, the mounting attachment 148 is removable. In other embodiments, the configuration of the mounting attachment 148 varies to accommodate mounting ports used in the various countries.

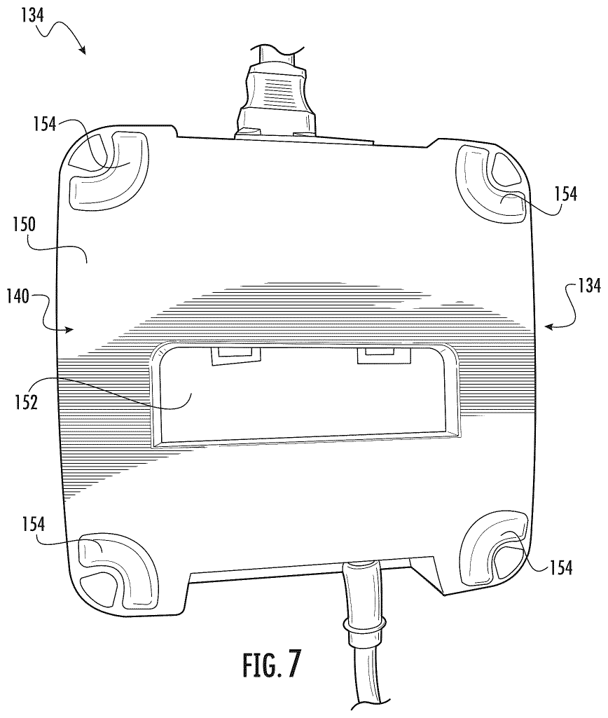

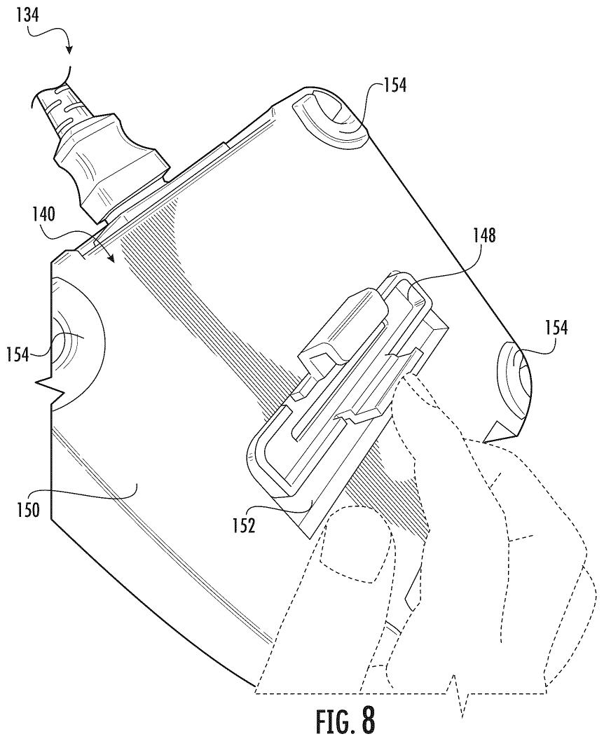

As shown in FIGS. 7 and 8, the pump system 134 includes stabilizing feet 154 (e.g., stands). The stabilizing feet 154 are positioned at each corner of the bottom face 150 and fused to the pump housing 140. The stabilizing feet 154 are configured to ensure the pump system 134 remains stable throughout operation. In some embodiments, the stabilizing feet 154 are configurable to shorten or lengthen to allow the pump system 134 to remain level in various environments (e.g., a flat surface, an inclined surface).

As shown in FIGS. 1-5, the pump system 134 includes a pump tube 132 (e.g., second tube). The pump tube 132 includes a pump tube first end 156 and a pump tube second end 158. The pump tube first end 156 is coupled to a pump inlet 142. The pump tube second end 158 is coupled to the pump port 130. The pump tube 132 is configured to create negative pressure (e.g., a vacuum, suction, suction pressure) within the cannister 116. The pump tube 132 facilitates flow of a fluid (e.g., air, gas) between the cannister 116 and the pump system 134.

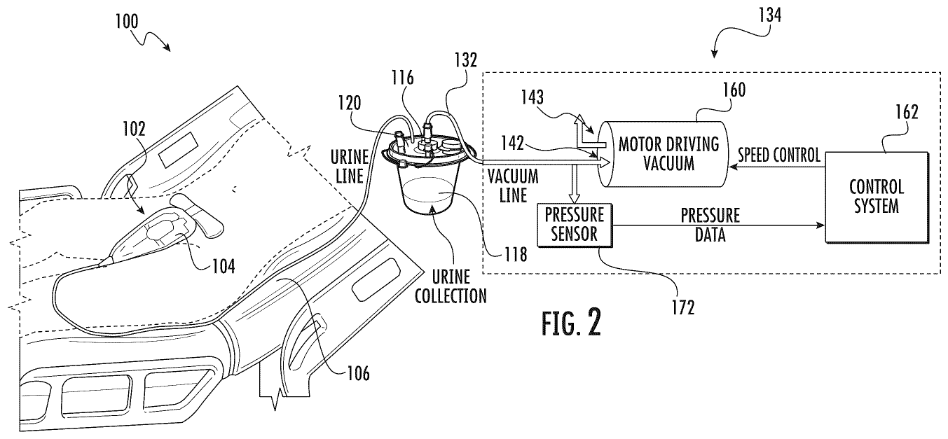

Referring back to FIGS. 1 and 2, the pump system 134 includes a motor 160. The motor 160 is an electric motor (e.g., direct current (DC) motor, alternating current (AC) motor). In some embodiments, the motor 160 is a brushless DC motor (BLDC) or a diaphragm pump motor. The motor 160 is positioned within the pump housing 140. The motor 160 is configured to drive the pump 141 to create (e.g., generate) suction pressure within the pump tube 132. For example, the control system 162 may instruct the motor 160 to generate the pressure of the fluid within the pump tube 132. A speed of the motor 160 affects the flow rate of air in the pump tube 132, which consequently affects the pressure in the pump tube 132. The speed of the motor 160 during various states of operation (e.g., active state, sleep state, obstruction state) is determined by a pressure reading, as described in more detail below.

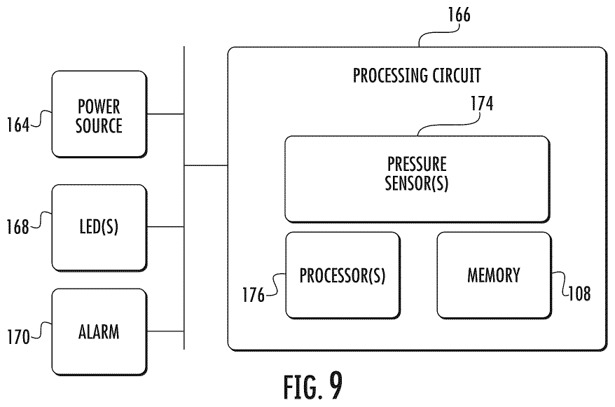

As shown in FIGS. 1 and 9, the pump system 134 includes a control system 162 (e.g., controller). The control system 162 is disposed in the pump housing 140. The control system 162 includes a power source 164, a processing circuit 166, visual indicators 168, and an alarm 170. The control system 162 is configured to control the operation of motor 160 and monitor the status of the patient via conditions of the pump tube 132 and cannister 116 (e.g., fill status, detection of obstructions).

The control system 162 includes a power source 164 (e.g., power supply). In some embodiments, the power source 164 includes a battery. As an example, the control system 162 may include a lithium-ion battery, a nickel-metal hydride battery, a nickel-cadmium battery, a lead-acid battery, a solid-state battery, a lithium polymer battery, or a similar medical-grade battery. In other embodiments, the urine management system 100 includes the power source 164 (e.g., the power source 164 is not part of the control system 162). For example, the control system 162 may include an electrical outlet. The power source 164 is configured to power the urine suction system 114.

The control system 162 includes a processing circuit 166. The processing circuit 166 includes one or more sensors 172, memory 174, and one or more processors 176. The processing circuit 166 is configured to (i) receive a signal from the one or more sensors 172, (ii) determine the pressure within the pump tube 132 based on the signal via the one or more processors 176, and (iii) compare the pressure to a pump threshold via the one or more processors 176.

The processing circuit 166 includes one or more sensors 172. The one or more sensors 172 are positioned within the pump tube 132 at the pump inlet 142. In some embodiments, the one or more sensors 172 are positioned on an exterior surface of the pump tube 132. In some embodiments, the one or more sensors 172 are positioned within the pump tube 132 at the pump port 130. As an example, the one or more sensors 172 include pressure sensors. The one or more sensors 172 are configured to measure pressure in the pump tube 132 near the pump inlet 142. In various embodiments, the one or more sensors 172 are configured to measure a signal associated with a pressure of the fluid within the pump tube 132.

The processing circuit 166 includes a memory 174 and one or more processors 176. The memory 174 may store instructions that, when executed by the one or more processors 176, cause the processing circuit 166 to perform various processes as described in more detail below.

The processing circuit 166 is configured to receive a signal associated with a pressure of the fluid within the pump tube 132 via one of the one or more sensors 172 (e.g., first sensor). The one or more sensors 172 are configured to measure pressure at the pump inlet through operation. In some embodiments, the one or more sensors 172 deliver a signal the pressure reading to the memory 174 and the one or more processors 176 continuously (e.g., every 1 second). In other embodiments, the one or more sensors 172 may be configured to deliver signals to the memory 174 and the one or more processors 176 at a configurable rate set by the nurse (e.g., every 5 seconds, every 30 seconds, every 1 minute, every 30 minutes, every 60 minutes, every 90 minutes).

The processing circuit 166 is configured to determine (e.g., calculate) the pressure based on the signal measured by the one or more sensors 172. The processing circuit 166 may evaluate signals detected by the one or more sensors 172 using the one or more processors 176. In some embodiments, the processing circuit 166 determines the pressure of the pump tube 132 at a given timestamp (e.g., the instantaneous pressure) using one or more sensors 172 during use of the urine management system 100. In some embodiments, the processing circuit 166 calculates an average of the pressure readings from the one or more sensors 172 within the pump tube 132 for a given timeframe during use of the urine management system 100. In other embodiments, the processing circuit 166, via the one or more processors 176, is configured to receive and calculate a change in pressure over time using signals from one or more sensors 172.

The processing circuit 166 is configured to compare the pressure to a pump threshold. The pump threshold is determined by a volume of the cannister 116 connected to the pump system 134. The processing circuit 166 compares the pressure reading to the pump threshold via the one or more processors 176. If the pressure reading is below (e.g., less than) the pump threshold, the processing circuit 166 is configured to cause the pump 141 to provide the fluid (e.g., air, gas) at a first rate through the pump tube 132. If the pressure reading is above (e.g., greater than) or the same as (e.g., equal to) the pump threshold, the processing circuit 166 is configured to cause the pump 141 to provide the fluid at a second rate through the pump tube 132. The second rate is greater than the first rate.

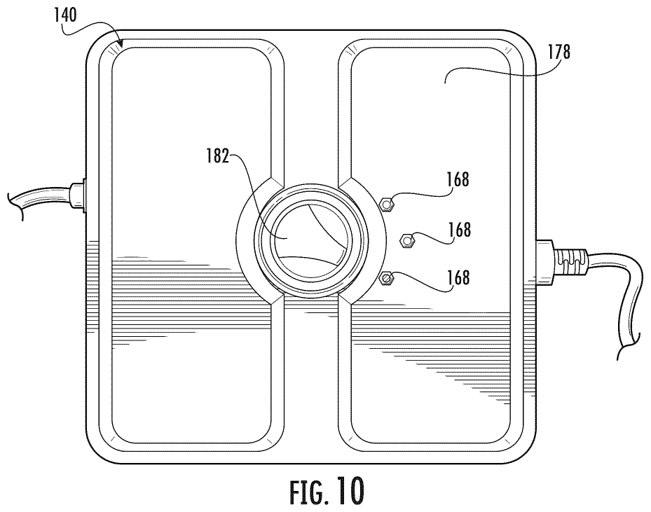

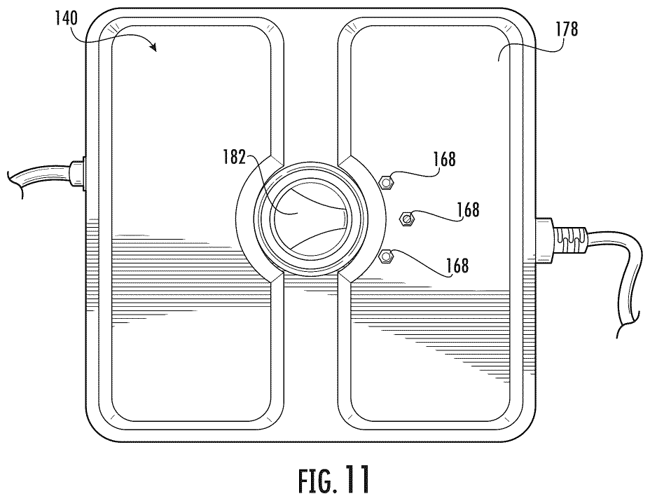

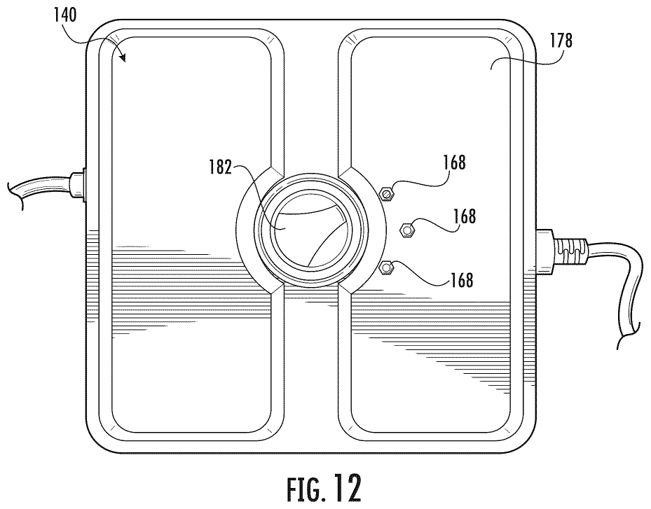

The control system 162 includes visual indicators 168. The visual indicators 168 are positioned on a top face 178 of the pump housing 140. In some embodiments, the visual indicators 168 are light emitting diodes (LEDs). The visual indicators 168 are positioned radially (e.g., around, surround) the knob 182. For example, as shown in FIGS. 10-12, the pump housing 140 may include three individual visual indicators 168. Each visual indicator 168 may have a different color (e.g., green, yellow, red) or other indication to denote between each state of the pump system 134 (e.g., active state, sleep state, obstruction state). The one or more processors 176 may transmit a signal to the visual indicators 168 to display a visual indication (e.g., flashing light sequence, color) to denote a current state of the pump system 134. In some embodiments, each visual indicator 168 displays a unique (e.g., different) visual indication. For example, as shown in FIG. 10, the pump housing 140 may include first visual indicator 168 (e.g., bottommost visual indicator) having a first visual indication such as a red, flashing light sequence to denote an obstruction is detected or the cannister 116 is filled to operational capacity. As another example, as shown in FIG. 11, the pump housing 140 may include a second visual indicator 168 (e.g., middle visual indicator) having a second visual indication such as a yellow, solid light to denote in a sleep state. As yet another example, as shown in FIG. 12, the pump housing 140 may include a third visual indicator 168 (e.g., topmost visual indicator) having a third visual indication such as a green, solid light or a green, flashing light sequence to denote in an active state. In some applications, the visual indicators 168 may be configured to notify a nurse when the pump system 134 is power on. In other applications, the visual indicators 168 may be configured to notify a nurse when a state of the pump system 134 has transitioned (e.g., changed) between a sleep state and active state. In yet another application, the visual indicators 168 may be configured to notify a nurse when an obstruction is present in the catheter tube 106.

The control system 162 includes an alarm 170. In some embodiments, the alarm 170 is disposed in the pump housing 140, in which a speaker is located on the pump housing 140. In some applications, the alarm 170 includes auditory signals (e.g., a beep, buzzer, warning sound, spoken warning given in preset language). In another application, the alarm 170 may include visual signals (e.g., flashing lights, warning symbol) displayed on a user interface of an external device (e.g., smartphone, a tablet, a laptop, a desktop computer stationed at a nurse or physician's station, pager, badge). In yet another application, after instructing the pump 141 to not provide the fluid, the control system 162 may transmit a signal to the alarm 170 over a network to an external device, in which the signal indicates a presence of an obstruction or the cannister 116 is filled to operational capacity. For example, the control system 162 may transmit a signal to the alarm 170 over a network such as Bluetooth, Bluetooth low-energy (BLE), Wi-Fi, near field communication (NFC), Internet of Things (IoT), or similar transmission signals to an external device. In yet another application, the control system 162 may transmit a signal to the alarm 170 to an external device with an antenna (e.g., bed, chair, patient monitoring system) in proximity to the urine management system 100 configured to relay signals to a main receiving device and/or receiving devices.

Referring now to FIGS. 1 and 10-12, the pump system 134 includes a knob 182. The knob 182 is positioned on the top face 178. For example, the knob 182 may be positioned on a central portion of the pump housing 140, equidistant from each respective edge of the pump housing 140. As another example, the knob 182 may be positioned along a respective edge of the top face 178. The knob 182 is configured to indicate the state of the pump system 134 (e.g., sleep state, active state, and obstruction state). The knob 182 is coupled to the motor 160, such that the motor 160 adjust the positioning of the knob 182. In some embodiments, the knob 182 dynamically adjusts its positioning to point towards a respective visual indicator 168 dependent on a signal from the control system 162. For example, as shown in FIG. 10, the control system 162 may deliver a signal to the motor 160 to prompt the knob 182 to point towards a first visual indicator 168 (e.g., bottommost visual indicator) if an obstruction is detected. As another example, as shown in FIG. 11, the control system 162 may transmit a signal to the motor 160 to prompt the knob 182 to point towards a second visual indicator 168 (e.g., middle visual indicator) if the pump system 134 is currently in a sleep state. As another example, as shown in FIG. 12, the control system 162 may transmit a signal to the motor 160 to prompt the knob 182 to point towards a third visual indicator 168 (e.g., topmost visual indicator) if the pump system 134 is currently in an active state.

In some embodiments, the knob 182 is configured to manually change the state of the pump system 134. The operator may engage with (e.g., turn, twist, push) or otherwise manipulate the knob 182 to prompt the control system 162 to transition between states (e.g., sleep state, active state, and obstruction state). For example, as the pump system 134 is operating in the active state, and providing the fluid at the second rate, the control system 162 may be configured to instruct the pump 141 to transition between providing the fluid at the second rate to providing the fluid at the first rate via an engagement of the knob 182 to point towards the second visual indicator 168 (e.g., middle visual indicator). As another example, as the pump system 134 is operating in the active state, and providing the fluid at the second rate, the control system 162 may be configured to instruct the pump 141 to transition between providing the fluid at the second rate to not providing fluid via an engagement of the knob 182 to point towards the first visual indicator 168 (e.g., bottommost visual indicator). As yet another example, as the pump system 134 is operating in the sleep state, and providing the fluid at the first rate, the control system 162 may be configured to instruct the pump 141 to transition between providing the fluid at the first rate to providing the fluid at the second rate via an engagement of the knob 182 to point towards the third visual indicator 168 (e.g., topmost visual indicator).

### III. Overview of Example Operation of Pump System

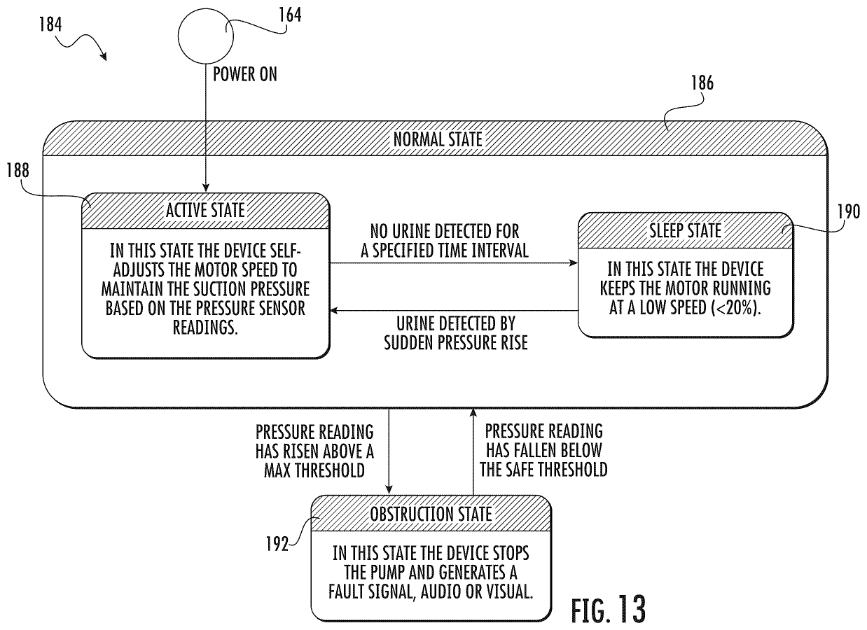

FIG. 13 illustrates a process 184 for the pump system 134. As previously mentioned above, the pump system 134 is configured to adjust suction pressure during various periods of use. The pump system 134 is configured to function in a normal state 186. The normal state 186 includes two states: an active state 188 and a sleep state 190. However, if a malfunction occurs (e.g., the cannister 116 is filled at or above operational capacity, an obstruction or blockage forms in the pump tube 132), the pump system 134 is configured to transition to an obstruction state 192 until the hazard has been resolved. It should be understood that the process 184 is not limited to the configurations as shown in FIGS. 13-16.

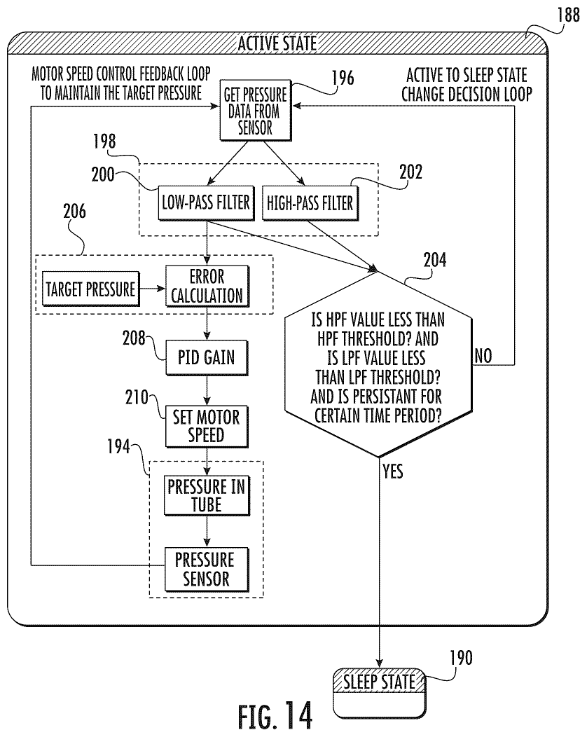

As shown in FIGS. 13 and 14, the process 184 includes the pump system 134 transitioning from an active state 188 and a sleep state 190 when urine is absent. When the pump system 134 is powered on with the power source 164, the pump system 134 begins in the active state 188. At act 194, the one or more sensors 172 detects pressure reading from the pump tube 132. At act 196, the one or more sensors 172 delivers the pressure reading to the processing circuit 166.

At act 198, the processing circuit 166 filters the signals detected by the one or more sensors 172 using the processing circuit 166. In various embodiments, the processing circuit 166 further include one or more low-pass filters 200 and one or more high-pass filters 202. The one or more low-pass filters 200 are configured to remove sudden fluctuations in the pressure reading, and the one or more high-pass filters 202 are configured to extract the sudden fluctuations from the pressure reading. In various embodiments, the pressure reading determines a low-pass pressure value and a high-pass pressure value (e.g., pressure values).

At act 204, the one or more processors 176 compare the pressure values to the pump threshold. In various embodiments, the pump threshold is determined by a low-pass threshold and a high-pass threshold. In various embodiments, the low-pass threshold and the high-pass threshold are different values. If (i) the low-pass pressure value is less than the low-pass threshold for a specified time threshold, and (ii) the high-pass pressure value is less than the high-pass threshold for a specified time interval, the pump system 134 transitions into the sleep state 190. The specified time interval may include a configurable amount of time (e.g., 30 seconds, 60 seconds, 90 seconds, 120 seconds). However, if the any one of conditions (i), (ii), or (iii) are not met, the pump system 134 continues to adjust the speed of the motor 160 to maintain suction pressure based on the pressure readings via the one or more processors 176.

At act 206, the control system 162 determines an error value based on an established target pressure. The error value is calculated by the difference between the measurement from the one or more processors 176 and the pump threshold. At act 208, the control system 162 inputs the error value into a Proportional-Integral-Derivative (PID) feedback control system strategy. The PID feedback control system strategy calculates the adjustments (e.g., changes) to be made to the speed of the motor 160 to maintain a target pressure during the active state 188. At act 210, the control system 162 instructs the motor 160 to adjust speed based on the calculations derived from the PID feedback control system.

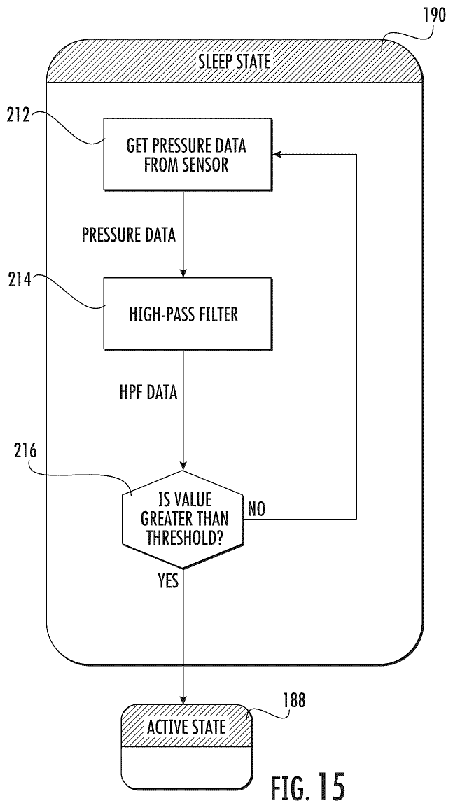

As shown in FIGS. 13 and 15, the process 184 includes the pump system 134 transitioning between sleep state 190 and active state 188 when urine is present. In sleep state 190, the motor 160 is set to a very low speed (e.g., the first rate). For example, the first rate may be less than 20% of operating capability of the pump system 134 (e.g., second rate). As another example, the first rate is between 10% and 30% of operating capability of the pump system 134 (e.g., the second rate). In act 212, the one or more sensors 172 detect a pressure reading from the pump tube 132 and deliver the pressure reading to the processing circuit 166. In act 214, the one or more high-pass filters 202 detects sudden fluctuations in the pressure readings over a specified time interval. In act 216, one or more processors 176 compare the pressure readings to the pump threshold. If the real-time high-pass filter pressure value is greater than the pump threshold (e.g., urine is detected causing pressure to increase), the pump system 134 transitions from a sleep state 190 to active state 188. If the real-time high-pass filter value is less than the high-pass threshold, the pump system 134 remains in the sleep state 190.

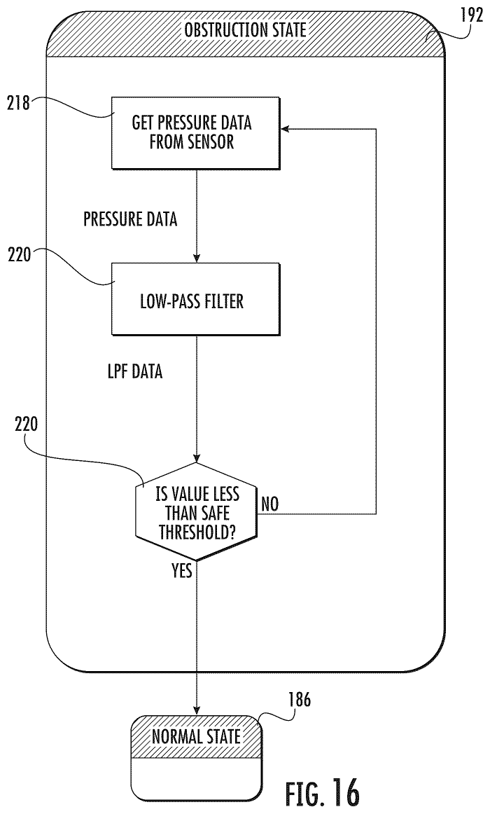

As shown in FIGS. 13 and 16, the process 184 includes the pump system 134 transitions between a normal state 186 and an obstruction state 192 when (i) the cannister 116 is filled to operational capacity and (ii) an obstruction is present in the pump system 134. If the cannister 116 is filled to operational capacity, the floating valve will stop the suction path and raise the suction pressure in the pump system 134 immediately to a very high value (e.g., a value 1.5× larger than the pump threshold, a value 2× larger than the pump threshold, a value 2.5× larger than the pump threshold). If the measured pressure value exceeds the maximum safe threshold, the pump system 134 switches to the obstruction state 192. In obstruction state 192, the motor 160 is turned off completely and triggers the alarm 170 to notify the nurse to empty the cannister 116 or resolve the obstruction. For example, after instructing the pump 141 to not provide the fluid, the control system 162 may transmit a signal to the alarm 170 and/or over a network to an external device, in which the signal indicates a presence of an obstruction or the cannister is filled to operational capacity. In some embodiments, the motor 160 remains idle to during the obstruction state 192 to protect the motor 160. In some embodiments, the motor 160 continues to run but at a significantly lower speed than of the sleep state 190. However, the control system 162 continues to iterate until the nurse resolves the hazard.

In act 218, the one or more sensors 172 detect a pressure reading from the pump tube 132 and deliver the pressure reading to the processing circuit 166. In act 220, the one or more low-pass filters 200 removes unwanted high frequency noise over a specified time interval. In act 222, the one or more processors 176 compare the pressure value to the maximum safe threshold. If the real-time low-pass filter pressure value is less than the maximum safe threshold (e.g., the cannister 116 is emptied or the obstruction is resolved), the pump system 134 transitions from an obstruction state 192 to normal state 186. If the real-time low-pass filter pressure value is greater than the maximum safe threshold, the pump system 134 remains in the obstruction state 192 until the nurse resolves the hazard.

### IV. Configuration of Example Embodiments

As utilized herein with respect to numerical ranges, the terms “approximately,” “about,” “substantially,” and similar terms generally mean +/−10% of the disclosed values, unless specified otherwise. As utilized herein with respect to structural features (e.g., to describe shape, size, orientation, direction, relative position), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “example” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other example embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general-purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an example embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can include RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general-purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A urine suction system comprising:
a cannister comprising a first port;
a first tube coupled to the first port and configured to receive fluid from the first port; and
a pump system comprising:
a pump inlet coupled to the first tube and configured to receive the fluid from the first tube,
a first sensor configured to provide a signal associated with a pressure of the fluid within the first tube,
a pump configured to receive the fluid from the pump inlet,
a pump outlet configured to receive the fluid from the pump, and
a processing circuit comprising a memory and one or more processors, the one or more processors configured to:
receive the signal from the first sensor,
determine the pressure based on the signal,
compare the pressure to a pump threshold,
instruct the pump to provide the fluid at a first rate when the pressure is less than the pump threshold, and
instruct the pump to provide the fluid at a second rate greater than the first rate when the pressure is greater than or equal to the pump threshold.

2. A urine management system comprising:
a catheter; and
the urine suction system of claim 1;
wherein the cannister further comprises a second port;
wherein the urine suction system further comprises a second tube coupled to the second port and the catheter;
wherein the second tube is configured to receive the fluid from the catheter; and
wherein the second port is configured to receive the fluid from the second tube.

3. The urine suction system of claim 1, wherein the first rate is between 10% of the second rate and 30% of the second rate.

4. The urine suction system of claim 1, wherein the pump system further comprises a floating valve between the pump inlet and the pump, the floating valve operable between a first position and a second position, the floating valve facilitating flow of the fluid from the pump inlet to the pump in the first position and prohibiting flow of the fluid from the pump inlet to the pump in the second position, the floating valve configured to be in the first position when the pressure is greater than or equal to a valve threshold, and the floating valve configured to be in the second position when the pressure is less than the valve threshold.

5. The urine suction system of claim 4, wherein the one or more processors are further configured to:
instruct the pump to not provide the fluid when the pressure is less than the pump threshold and the floating valve is in the second position.

6. The urine suction system of claim 5, wherein:
the pump system further comprises a motor coupled to the pump, the motor configured to drive the pump to create suction pressure within the first tube; and
the motor is configured to remain idle when the floating valve is in the second position.

7. The urine suction system of claim 6, wherein:
the pump system further comprises an alarm coupled to the processing circuit; and
the one or more processors are further configured to:
after instructing the pump to not provide the fluid, cause the alarm to transmit a second signal over a network to an external device, the second signal indicating a presence of an obstruction or the cannister is filled to operational capacity.

8. The urine suction system of claim 1, wherein the first sensor is positioned within the first tube at the pump inlet.

9. The urine suction system of claim 1, wherein the pump is enclosed within a pump housing, the pump outlet and the pump inlet positioned on a first face of the pump housing.

10. The urine suction system of claim 9, wherein:
the pump housing further comprises a first visual indicator and a second visual indicator positioned on a top face of the pump housing; and
the one or more processors are further configured to:
after instructing the pump to provide the fluid at the first rate, provide a first indication via the first visual indicator, and
after instructing the pump to provide the fluid at the second rate, provide a second indication via the second visual indicator.

11. The urine suction system of claim 10, wherein:
the pump system further comprises a knob positioned on the top face; and
the one or more processors are further configured to:
instruct the pump to transition between providing the fluid at the second rate to providing the fluid at the first rate via an engagement of the knob.

12. The urine suction system of claim 10, wherein:
the pump system further comprises:
a knob positioned on the top face, and
a motor coupled to the pump and the knob; and
the one or more processors are further configured to:
after instructing the pump to provide the fluid at the first rate, instruct the motor to adjust the knob to a first position, a portion of the knob facing the first visual indicator in the first position, and
after instructing the pump to provide the fluid at the second rate, instruct the motor to adjust the knob to a second position, the portion facing the second visual indicator in the second position.

13. The urine suction system of claim 1, wherein the one or more processors are further configured to:
instruct the pump to transition from providing fluid at the second rate to providing fluid at the first rate when the pressure is less than the pump threshold for a specified time interval.

14. The urine suction system of claim 1, wherein:
the pump system further comprises a floating valve between the pump inlet and the pump, the floating valve operable between a first position and a second position, the floating valve facilitating flow of the fluid from the pump inlet to the pump in the first position and prohibiting flow of the fluid from the pump inlet to the pump in the second position, the floating valve configured to be in the first position when the pressure is greater than or equal to a valve threshold, and the floating valve configured to be in the second position when the pressure is less than the valve threshold; and
the one or more processors are further configured to instruct the pump to not provide the fluid when the pressure is less than the pump threshold and the floating valve is in the second position.

15. A method of operating a urine suction system, the method comprising:
receiving, by one or more processors, a signal from a first sensor, the signal associated with a pressure of a fluid within a pump tube coupled to a cannister and a pump;
determining, by the one or more processors, a pressure value based on the signal;
comparing, by the one or more processors, the pressure value to a pump threshold;
when the pressure value is less than the pump threshold, instructing, by the one or more processors, the pump to provide the fluid at a first rate via the pump tube; and
when the pressure value is greater than or equal to the pump threshold, instructing, by the one or more processors, the pump to provide the fluid at a second rate greater than the first rate via the pump tube.

16. The method of claim 15, further comprising determining the first rate based on the second rate;
wherein the first rate is between 10% of the second rate and 30% of the second rate.

17. The method of claim 15, further comprising instructing the pump to provide the fluid at the first rate or the second rate via the pump tube only when a floating valve positioned within the pump tube is in a first position.

18. The method of claim 17, further comprising instructing the pump to not provide the fluid when the pressure is less than the pump threshold and the floating valve is in a second position, the second position different than the first position.

19. The method of claim 18, further comprising instructing the pump to transition from providing fluid at the second rate to providing fluid at the first rate when the pressure is less than the pump threshold for a specified time interval.

20. The method of claim 15, further comprising:
instructing a motor coupled to the pump to generate the pressure of the fluid within the pump tube; and
instructing the motor to adjust the pressure of the fluid such that the pump provides the fluid at the first rate via the pump tube.
